(19) United States
(12) Patent Application Publication
(10) Pub. No.: US 2014/0018636 A1
(43) Pub. Date: Jan. 16, 2014

(54) DYNAMIC SCALE AND ACCURATE FOOD MEASURING

(71) Applicants: Oliver M. Contant, Redmond, WA (US); Christine V. Contant, Redmond, WA (US)

(72) Inventors: Oliver M. Contant, Redmond, WA (US); Christine V. Contant, Redmond, WA (US)

(21) Appl. No.: 13/881,687

(22) PCT Filed: Jun. 28, 2011
(86) PCT No.: PCT/US11/42103

§ 371 Date: Sep. 27, 2013

Related U.S. Application Data
(60) Provisional application No. 61/359,378, filed on Jun. 29, 2010.

(51) Int. Cl. A61B 5/00; A61F 13/42; A61F 5/48; A61B 5/11; G01G 17/00

(52) U.S. Cl. CPC A61B 5/4866; A61B 5/0077; A61B 5/686; A61B 5/1112; A61B 5/4812; A61B 5/1118; A61B 5/486; G01G 17/00; A61F 5/48; A61F 13/42
USPC 600/301; 600/300; 600/476; 600/595

(57) ABSTRACT

A Health Management System that can assist a user in measuring and regulating his or her dietary intake and optionally his or her physical activity is disclosed.

### CROSS-REFERENCE TO RELATED APPLICATIONS

This application is related to U.S. Provisional Patent Application No. 61/359,378 entitled “Dynamic Scale and Accurate Food Measuring,” and filed on Jun. 29, 2010, which is hereby incorporated by reference.

### BACKGROUND

Health is a constant concern for the human population. Even outside of specific diseases, healthy living can promote well-being, energy, and a long life. Many people worry daily about weight management, proper exercise, proper diet, and other health related issues. Numerous government organizations (e.g., the World Health Organization and Center for Disease Control) and publications are dedicated to informing people about the role of their activities in increasing health and wellness. Many corporations have developed wellness programs to encourage workers to maintain or improve their health. Health encompasses physical, mental, and emotional factors and affects many areas of a person's life.

There are many existing methods for monitoring and improving health. Individual devices exist for measuring various indicators of health, such as heart rate monitors, blood pressure monitors, glucose tests, and so forth. Many devices exist that are related to fitness, such as exercise bicycles, treadmills, weights, and so forth. In addition, many systems exist for managing a person's diet, such as counting calories, the Atkins diet, and so forth. Some programs will send meals to a person that are designed to deliver a specific amount of calories per day.

Unfortunately, overall health is difficult to track, and many existing systems integrate poorly with a person's lifestyle. For example, monitoring devices are only helpful if the person remembers to use them and record their results, diets are only as good as the person's discipline to follow the diet's rules, and other elements in a person's life may add to or detract from health that current systems do not track or consider at all. For example, the number of hours a day that a person watches television, whether the person drives or bicycles to work, and even the speed at which a person eats can all impact the person's health. In addition, even when useful health data is collected about a person, it is difficult for the person to act on that data. The person may invest significant hours learning about a number of calories suited to that person's lifestyle, the amount of exercise that person needs, or the types of food he or she should eat.

### SUMMARY

Health management systems such as dietary intake regulating devices that can assist a user in measuring his or her dietary intake and optionally his or her physical or sleep activities are disclosed.

In one embodiment, the health management system comprises various types of sensors (such as a camera) for identifying the device user (e.g., for authentication purposes), for detecting the load or unload of food from the device (e.g., for regulating eating speed), for identifying the food characteristics (e.g., for later review and analysis by a physician), for recording the device geographical location (e.g., for evaluating if user ate in a junk food restaurant), for detecting user's sleep stage cycles (e.g., for waking up a child with bedwetting issues when the device detects he is entering the NREM stage 1), or for monitoring when the device is motionless while the user is eating with the device (e.g., for regulating the user's eating pace).

In one aspect of the present invention, the health management system is configured as a weighting device that can tare itself or weigh a load on the device even when the device is moving. This functionality detects and compensates the load motion and therefore provides non-erroneous scale readings. Furthermore, the health management system is configured to calculate and provide in real-time the recommended user's energy balance (such as calorie intake and outtake) based on monitored data, which provides a more accurate recommendation critical for weight management.

In another aspect of the present invention, the healthy management system is configured with a labeling functionality and a bookmark functionality to identify or bookmark an activity/behavior and data associated with the activity/behavior. The system is also configured to alert the user of events using dynamic and adaptive reminders, allowing users to create reminders not only based on time but also based on events that will later be detected by device.

### BRIEF DESCRIPTION OF THE DRAWINGS

Preferred and alternative embodiments of the present invention are described in detail below with reference to the following drawings. These depict particular embodiments of the invention and are not intended to limit the scope of the invention. All of the drawings are schematics rather than precise representations and are not drawn to scale.

FIG. 1 is a schematic representation illustrating one exemplary configuration of the health management system including a dietary intake regulating device comprised of an enclosure, a spoon extension and a tooth extension, in accordance with the present invention; and

FIG. 2 is a block diagram of an exemplary enclosure, in accordance with the present invention.

### DETAILED DESCRIPTION

### Overview

A health management system 10 is described herein that includes, for example, an electronic utensil 22 for eating food and measuring health information. The system senses human activity using a variety of different methods and sensors 35, and records information for concurrent or subsequent analysis. For example, the system may capture information about the user's caloric intake, eating schedule, pictures of foods eaten, and so forth, and present the user with historical records and a plan for healthy living.

### User Identification

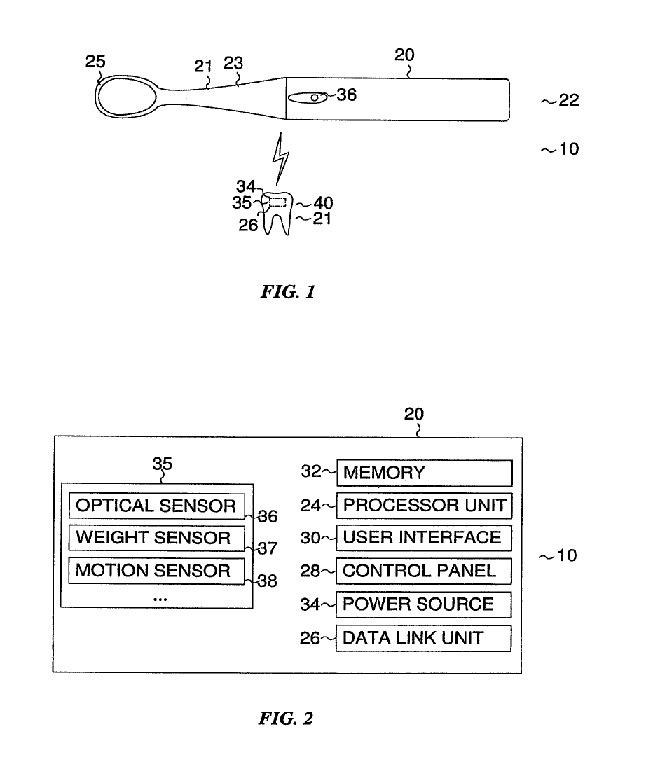

The health management system 10 may identify a user in a variety of ways, such as by using the optical device 36 (i.e., a camera in FIG. 1) described herein and face recognition software. In some embodiments, the device is configured to read one or more fingerprints of the user, e.g., to identify a user, to personalize the user's data, or to grant access to a pre-determined set of device functionalities. The device may be (or may be integrated into) any type of device, e.g., an eating utensil, a food carrier, a monitoring device such as a physical activity monitoring device or a non-activity monitoring device (such as a sleep monitoring device), or a blood glucose meter device.

### Camera-Based Detection

In some embodiments, the health management system 10 includes a device capable of recording images (e.g., using a digital camera module) when a user loads/unloads food on/from a food carrier 25, such as a fork, spoon 23, chopstick, human hand, or finger. The device can accurately detect when food is present or not present on the food carrier 25. For example, the device can detect when a user loads food on a food carrier 25 from his plate between bites and when the user unloads food in his mouth. The device can be located (i) on the food carrier 25 or on a food carrier extension 21 (e.g., attached or integrated) or (ii) in the proximity of the food carrier 25, with the focus of the device pointing on the food carrier to accurately capture images. The process to infer the load or unload can be based, for example, on known or future image processing technology. FIG. 1 illustrates an enclosure 20 with a camera 36 pointing to the spoon 23 (i.e., an extension 21) attached to the food carrier 25 and communicating with a second extension 21, i.e., a dental crown 40. The dental crown 40 in FIG. 1 is shown with a power source 34, one or more sensors 35 and a data link unit 26.

The device may further include one or more additional recording capabilities to record not only the food or the load/unload of food but also other correlated events, such as taking a picture of the food carrier user by having the camera pointing at the user's face, or taking a picture of a glass or drink that are next to a plate by having the camera pointing at the user's plate (using a wide angle lens if needed). For example, the device may accurately record the user's face image by using (i) a wide-angle lens to provide a wide-angle view and (ii) a linear and angular accelerometer to detect the orientation of the food carrier and the camera to record the image during an upward movement of the food carrier to the user's mouth. The device is configured to record images when the relevant event is identified, e.g., by automatically positioning the camera toward the desired target and then taking a large angle picture, or, e.g., by detecting while the device is moving when the device is automatically focused on the desired target and then taking a large angle picture.

In some embodiments, the device is further configured to record images of the food that the user is eating. For example, the device may detect the up/down movement of the food carrier 25 (e.g., via an accelerometer or gyro), and take a picture of the plate containing the food during the down movement. The device may also detect a food load event on the food carrier and take a picture of the food upon detecting that event. Similarly the device is configured to record images of liquids that the user is drinking, e.g., during a meal. These images are then processed for further analysis, for example, to detect the characteristics of the food or liquid (e.g., levels of fat, carbohydrate and protein, portion size, junk or healthy food categorization, high carbohydrate drink, and so on). This analysis may be directly processed by, e.g., using database and image processing techniques, or indirectly processed by, e.g., sending information to a server that will transmit this information to a relevant third party for further processing.

Moreover, the device can trigger the recording of images on-demand, e.g., by triggering a button or a touch screen, or automatically (in a random fashion or not), e.g., at a time-related event, at an activity generated event, at a position generated event, or at the device detection of an object or a mass present on the food carrier via a proximity or strain sensor, or, e.g., taking a picture of food when a weight is detected on the food carrier, or, e.g., taking a picture of the user's face when a bite is detected, when the food is unloaded in the mouth or when the utensil is approaching the plate.

In some embodiments, the health management system 10 groups, associates, and/or tags images based on a common event. For example, the system may assign the same tag to all images that are recorded within a period or at a similar location. In this example, the detection of the meal event can be performed, e.g., using a utensil that can detect the beginning and the end of a meal by identifying the first and last bite of meal. These image associations allow the user or a 3rd party to comprehensively assess a meal and its environment, e.g., by identifying the images, such as the number of bites during the meal, the characteristics of the food, the characteristics of the drink, and the user identity.

In some embodiments, the device optical sensor 36 (such as a camera) may replace several other common sensors, such as a proximity sensor, a motion sensor 38, or a bite detection sensor. In addition, the device can be configured to receive different types of optical filters or lenses, such as, e.g., infrared filters, for infrared thermography of food or liquid, thermal imaging or thermal video, or, e.g., polarizing filters, for example linear polarizer and linear focus typically used on non-auto focus cameras, or, e.g., wide angle conversion lenses to provide wide angle views. Therefore the device is further capable of identifying other parameters based on the filter or lens capabilities, such as, e.g., the temperature of food or focusing without an auto focus feature.

In some embodiments, the device may include a temperature sensor or infrared filter for measuring the temperature of an object on the food carrier or on the eating utensil tip, e.g., measuring the food temperature on a fork or the soup temperature on a spoon. Temperature information may be used during analysis of likely digestion and caloric processing of the food by the user's body.

Referring now to FIG. 2, a block diagram representation of an exemplary electric circuit for inclusion in the health management system 10 or an exemplary enclosure 20, in accordance with an embodiment of the present invention, is shown. The electric circuit or the enclosure 20 in the illustrated embodiment include a processor unit 24, a data link unit 26, a control panel 28, a user interface 30, a memory unit 32 and a power source 34. The electric circuit or the enclosure 20 also include a plurality of sensors 35 such as an optical sensor 36, a weight sensor 37 and a motion sensor 38. However, it should be understood that components can be added to or removed from the electric circuit or the enclosure 20 or otherwise changed without departing from the scope of the present invention.

### Weight Detection

In some embodiments, the health management system 10 includes a weight detection sensor 37 capable of measuring the weight of an object eaten by a user even if/when the object is in motion, e.g., measuring the amount of food on a food carrier while the food carrier is moving. Motion and gravity are two phenomena that affect accurate weight measurements. Therefore, this device has several specific functionalities described herein.

Many scales have a tare function or “tare weight” setting, which allows the user to hit the tare button to zero the scale out (and this even if an empty container is on the scale). Moreover scales need to be reset to zero often (if not periodically) to continue offering accurate measurements. If the scale is moving then the tare function cannot work due to gravity and motion. The first functionality of the device is to automatically reset the scale to zero (commonly known as tare) as soon as the device detects that it is not in motion, e.g., using a motion sensor.

Measuring a weight on a scale that is in motion (for example, an eating utensil that has a scale on the food carrier part) is difficult since movements create linear or angular accelerations and the weight measurement readings are therefore impacted by such forces. For example, moving up a food carrier 25 on a vertical axis by 2 g will double the weight measurement on the food carrier. The second device functionality is to automatically weigh an object or mass on a scale as soon as the device detects that it is not in motion, e.g., using a motion sensor. This second device functionality may use the same principles as the ones used in the first device functionality.

The third device functionality is to weigh an object or mass while in motion by dynamically compensating for the detected device motion and the associated forces. This device can therefore be considered as, for example, an adaptive and dynamic weighting scale that reads the linear and angular accelerations of the device to compensate on the fly the reading on the scale and therefore infer the correct weight as if the scale was not moving.

The fourth functionality of the device is to automatically reset the scale to zero while the device is in motion by dynamically compensating for the detected device motion and the associated forces. This fourth functionality may use the same principles as the ones used in the third device functionality.

Applying the tare functionality or measuring the weight of an object or mass is only valid if you know that the object or mass you want to weight is present or not on the scale. Therefore, a scale or third party detects the presence or absence of the entity to be weighted in order to automatically trigger the tare functionality or to automatically weigh food. The fifth device functionality is to detect if the intended object or mass to be weighted is present or absent on the scale. For example, if the object is present on the scale then the device can measure the weight of the object, and if the object is absent on the scale then the device can reset the scale to zero (tare). As another example, the device may be a food carrier 25 such as a spoon 23 with an integrated scale on the tip of the spoon and with an integrated camera 36 that detects food presence or absence. As another example, the absence of food on the spoon 23 can be inferred by detecting the user's bites, i.e., weighing food before the user takes a bite and resetting the tare right after the user takes a bite. The presence or absence of the intended object or mass to be weighed can be detected by known or unknown detection sensors or systems, such as, e.g., weight 37, optical 36 or proximity sensors, or bite detection systems.

Any combination of the device functionalities can therefore be integrated in the device to accurately tare the device (i.e., zero out the scale) or weigh an object or mass. The device motion such as, e.g., the linear or angular accelerations, speed or distance, and the forces applied to the device such as, e.g., torsion or gravitational forces, can be detected using known or future sensors, such as, e.g., accelerometers or gyros which are two known motion sensors 38. The device may be (or may be integrated into) any type of device, e.g., an eating utensil, a food carrier 25, a monitoring device such as a physical activity monitoring device or a non-activity monitoring device (such as a sleep monitoring device), or a blood glucose meter device.

### Location Detection

In some embodiments, the health management system 10 includes a device configured to (i) record its location or (ii) infer a place where the device is located, both during a food intake event (such as, e.g., meal time, snack time, the starting or end of a meal, a specific time elapsed since the previous meal, a specific time remaining until a next scheduled meal, or taking a bite of food), or/and during an activity event (such as, e.g., exercising, jogging, the beginning or end of an activity), or any combination. The system may use the location, e.g., to infer a restaurant location and type of food served in the restaurant, or to infer a place where the user exercised. This information can later be used, e.g., to change unhealthy behaviors of the user by recommending better restaurants or places to eat, or recommending better places to exercise if the place is unhealthy (for example if running on city streets). The geographical location can be monitored using known or unknown systems, such as, e.g., a global positioning system (GPS) receiver unit, a system that applies triangulations or field strength calculations to determine the position of the device (e.g., a mobile phone), or a system that applies a MAC address of a base unit in a wireless network to determine a position of the device.

For privacy reasons, the device may be configured to limit the amount of information recorded, e.g., the device may only allow the system to extract a name associated to the location, e.g., by only allowing to record and share the restaurant name without its exact location.

In some embodiments, the user can configure the system to only store a flag or a grading system automatically capturing the healthiness of the location the user eats. For example, eating at home is 5/5, eating at Whole Foods is 5/5, eating at a fast food restaurant is 1/5, and so forth, where 1 is the least health and 5 is the most healthy. The device may be (or may be integrated into) any type of device, e.g., an eating utensil, a food carrier, a monitoring device such as a physical activity monitoring device or a non-activity monitoring device (such as a sleep monitoring device), or a blood glucose meter device.

Alternatively or additionally, the device may not be directly configured to locate the user's position or to identify the location name but may only do it indirectly via a 3rd party, i.e., e.g., the device may be configured to send a request to a 3rd party device in its proximity that is configured to locate the user's location and to reply to the request with the location data or location name. Therefore, the device can directly or indirectly capture the location of a user.

### Information Detection

In some embodiments, the health management system 10 includes a device configured to automatically scan/read food or liquid information on a product such as, e.g., a label on a food or liquid package, a bar code on a product, a bar code on a restaurant menu item, or any other available product-dependent indication. The device is further configured to automatically extract, record, and categorize detected information. The system analyzes the information to automatically extract additional information (e.g., food ingredients, food quantities, or food warnings) directly (e.g., from the scan) or indirectly (e.g., by transmitting the scanned information to a server or an internal database that includes detailed information on the product).

The device may be (or may be integrated into) any type of device, e.g., an eating utensil, a food carrier, a monitoring device such as a physical activity monitoring device or a non-activity monitoring device (such as a sleep monitoring device), or a blood glucose meter device. The device scanning technology may be any known or unknown scanning process such as, e.g., pen-type reader, image scanner, laser scanner, CCD reader, camera-based reader, barcode scanner, or cell phone camera.

A detected food or liquid information can thereafter be integrated in a database and associated with other related information. For example, the device may be a fork that detected the beginning of a meal, detected the load and unload of food and took picture of the food when food was loaded for further processing, detected the location and the name of the place where the user is eating; then the device reads the barcode of the menu item in a restaurant and associates information extracted from the barcode to all other related-information recorded, i.e., the time stamps of the beginning of the meal, of each bite, the picture of the food when loaded on the fork, the weight of food after each bite, and so on.

### Sleep Detection

In some embodiments, the health management system 10 includes a device configured to detect sleep cycles of a user using sensors 35 (such as a motion sensor 38) and to randomly or automatically trigger a signal or an alarm when the user is reaching a specific phase in his sleep cycles. This device therefore allows waking up a user, e.g., only when the user reaches the less disturbing phase of his/her sleep cycles (e.g., phase one), or when the user reaches a sleep phase (e.g., phase four) where issues (e.g., sleep terror, somnambulism or enuresis) can occur, or when an interesting event (e.g., sleep terror, somnambulism or enuresis) is detected via sensors, or any combination thereof.

Sleep begins in stage one of the sleep phases known as non-rapid eye movement (NREM) sleep. NREM sleep has four stages: light sleep (stage one), deeper sleep (stage two), and two stages of deepest sleep (stage three and four). Stage one is the “drifting off” period of light sleep in the transition between wakefulness and sleep and comprises about five percent of the entire sleep period. Stage two involves a change in brain-wave patterns and increased resistance to arousal and accounts for 45-55 percent of total sleep time. Stages three and four are the deepest levels of sleep and occur only in the first third of the sleep period. NREM stage four usually takes up 12 to 15 percent of total sleep time. Sleep terrors, sleep walking, and bedwetting episodes generally occur within stage four or during partial arousals from this sleep stage. It typically takes about 90 minutes to cycle through the four deepening stages of NREM sleep before onset of the second phase of sleep known as REM or dream sleep.

Enuresis, the medical name for bedwetting, is an issue that millions of families face every night and is a common problem in kids, especially children under the age of 6 years. About 13% of 6-year-olds wet the bed, while about 5% of 10-year-olds do. Sleepwalking, also known as somnambulism, is a sleep disorder belonging to the parasomnia family. Sleepwalkers arise from the slow wave sleep stage in a state of low consciousness and perform activities that are usually performed during the day. As an example, the chronological order of the phases of a typical sleep cycle is as follows: the first hour of sleep: awake, stages 1/2/3/4; the second hour: stages 4/3/2/1, REM; the 3rd hour: REM, stages 1/2/3/4; the 4th hour: stages 4/3/2/1, REM; the 5th hour: REM, stages 1/2/3/2/1, REM; the 6th hour: REM, stages 1/2/1, REM; the 7th hour: REM, stages 1/2; the 8th hour: stages 2/1, awake.

The device can be configured to signal or alert the user (e.g., to wake up the user) (i) when the sleep cycle reaches a specific stage (e.g., stage 1 or stage 4), or (ii) when the sleep cycle does not follow its typical, usual, or expected cycle (e.g., due to a nightmare or night terrors, due to sleepwalking or somnambulism, due to enuresis or a urinating accident or bedwetting, and so forth), or any combination thereof. For example, the health management system 10 can help to change the behavior of a child to avoid urinating in bed by waking the child up each time the stage 1 phase is reached. In this example, the device can alert the child when each stage 1 is reached and this until the child press a “cancel/stop” button on the device to indicate that he went to the bathroom and does not need to be awake again during the night. Moreover the device can be configured to have an automatic “cancel/stop” functionality, e.g., by detecting the user's motion (e.g., via an accelerometer) while going to the bathroom and stopping the alert functionality automatically for the rest of the sleep cycle. Alternatively or additionally, the device can be configured to start the alert process only a certain time after the start of the sleep cycle, e.g., 1 hour or 1.5 hours after the child went to bed.

The device can also be configured to detect an uncommon event occurring in a sleep cycle by (i) detecting the current sleep cycle phase of the user and (ii) detecting any variation of the actual phase activity with a normal phase activity. Therefore the device can detect (using known or future sensors such as, e.g., a sleep meter) that the user is in a specific sleep stage (e.g., within sleep stage 4) and that an abnormal activity (e.g., a nightmare or night terrors, sleepwalking or somnambulism, enuresis or a urinating accident or bedwetting, etc.) is occurring by detecting (using known or future sensors such as, e.g., a sleep meter, a motion meter, an audible meter, a humidity sensor, or a proximity meter) one or more abnormal events (e.g., a partial arousal of a sleep stage (typically stage 4) when not expected, or a sudden movement); and then send an alert or a signal, e.g., to wake up the user or a 3rd party (e.g., a parent).

In some embodiments, the device includes a learning function to learn when a specific event reoccurs frequently while sleeping (e.g., a nightmare or night terrors, sleepwalking or somnambulism, enuresis or a urinating accident or bedwetting, and so on) which, e.g., is useful to more precisely optimize the alert or signal functionality. For example, the device can (directly or indirectly) detect in the sleep cycle some violent movements (e.g., using an accelerometer) that correspond to nightmares or night terrors and record every night at what time the events occur. After a test period, e.g., one week of normal sleep and recording, the system can adapt the alerts and reminders based on the test period to provide alerts that are more accurate or reminders. As another example, the device can (directly or indirectly) detect if a diaper or a fabric is humid (e.g., using a humidity sensor) and record every night at what time it happens and after a test period, e.g., one week of normal sleep and recording, adapt the alerts and reminders based on the test period to provide more accurate alerts or reminders. Therefore this device can also be configured to detect the humidity of a fabric (e.g., cloth, pajamas, diapers) using a humidity sensor to signal or alert when a humidity threshold is reached.

Furthermore the device can also be configured to detect a sleep activity period that does not correspond to a typical sleep activity (e.g., sleep walking, sleep terror or when an individual has a nightmare and moves or vociferates violently) via sensors (e.g., motion sensors like accelerometer or audio sensor like microphone), e.g., to send an alert or a reminder. Any combination of the described device functionalities can be implemented in the device. For example, the device may detect when a specific phase is reached and how many times it was reached since the user sleeps while also detecting humidity threshold or motion to correlate the information and provide accurate detection events (i.e., e.g., sleepwalking, sleep terrors, or bedwetting). The device may be (or may be integrated into) any type of device, e.g., a watch, a fabric, a diaper, an eating utensil, a food carrier, a monitoring device such as a physical activity monitoring device or a non-activity monitoring device (such as a sleep monitoring device), or a blood glucose meter device. The reminders or alerts can be transmitted via signals such as, e.g., visible, vibration or audible signals, or via known or unknown message carrier, such as, e.g., email, SMS, web, chat, tweeter message, instant message, or phone call, or any combination thereof. Reminders or alerts can be sent to the device user or to a third party, such as, e.g., a server, a smartphone, a speaker, a relative, or a parent to inform a 3^{rd} party of the signal or alert. This will allow, for example, a relative (such as a parent or husband) of the device user (such as child or wife) to be aware of or to supervise or to act upon the signal or alert if needed. Therefore, by communicating the signal or alert from the device to a 3^{rd} party, a parent could help awake a child to go to the toilets and a husband could help his wife to not hurt herself while she is sleep walking.

### User Behavior Modification

In some embodiments, the device is configured to pace the eating speed of a user by notifying the user (i) to put back the device for a period on a non-moving surface between each bite or (ii) to not touch the device for a period between each bite, or any combination thereof. The device can detect when the device is not in motion (e.g., using a motion sensor), start a timer and send a signal (e.g., audible, visible or vibration signal) when the pre-determined time period is reached to alert the user that he/she can take a next bite. The device can also detect when the device is in motion to record an amount of time the device is moving and send an alert (e.g., audible, visible or vibration signal) if moving more than a pre-determined time period. Moreover, the device can detect when a user is touching (or holding or carrying) the device (e.g., by using a touch sensor or a capacitance sensor on the food carrier part that is held by the user) to measure a period the device is held and to send an alert after a pre-determined period.

In some embodiments, a device used as a food carrier 25 or as an eating utensil is configured to regulate or detect the number of bites by detecting the non-movement of the device. The detection of non-movement can be based on a motion sensor 38 such as, e.g., an accelerometer, that is integrated or attached to the device. As soon as the sensor detects that the device is not moving, i.e., a non-movement event, then a timer starts and sends a signal to the user on the device or on a 3rd party device via a communication link. The signal can be, e.g., a tactile signal (a vibration), a visual signal (a series of LEDs) or an audible signal (a series of beeps). The timer may be dynamic and therefore the length of time or of the signal emission can augment or increase based on settings. Concurrently, the device records (in an internal memory 32 or in an external memory via a communication link) the date and time stamps of the events for later, processing or analysis. The non-motion detection can be also coupled with an angular sensor to detect not only the non-motion but also the angle of the device to trigger a non-motion event only when the device is in a specific orientation.

As one example, a user can use a fork implementing the health management system 10 to eat a meal and can be alerted to put down the fork next to the plate between each bite to start the bite detection/regulation process. Once the fork/device is down the sensor detects the non-motion event of the device and a series of 10 LEDs located on the device start to light up one after one till the 10 LEDs are all light up, indicating to the user that it is time to take a next bite. The device communicates the data events and date/time stamps to a server and the user can later access his device history events on a smartphone application or a website.

In some embodiments the device may be configured to receive the input from a user after each bite (such as, e.g., pressing a button or a touch screen on the device after each bite), or to pace the eating speed of a user (such as, e.g., a bite frequency timer that informs when to take the next bite), or any combination thereof. For example, the device can be an eating utensil or a food carrier with an integrated timer and a user-input component. The user can press a button after each bite to reset the timer and record his bite timestamp. The timer may include several preset alerts after specific time intervals (e.g., during the first 10 seconds the utensil displays a red warning or sends a vibration or plays an audible sound to prevent the user from taking a next bite, then it shows for 5 seconds an orange warning showing that the user will soon be able to take a next bite, and then a green light indicating the user can take a next bite). While waiting between two bites the device can provide tips or messages such as, e.g., chew or take a deep breath.

In some embodiments, the device is configured to inform the user of his/her eating rate (e.g., how fast or how slow he eats) directly on the device or indirectly on a third party device using a communication link. The eating rate can be inferred, e.g., from an automatic bite detection system located on the device or from the manual triggering of a hardware component located on the device (such as, e.g., a button or touch screen) or from a third party that communicates with the device. The information about the user's eating rate is provided on the device as a visual, audible, or sensual (such as vibration) signals, or any combination thereof. For example the eating pace of a user can be conveyed via an arrow going up or down to show the user is eating too fast or with a green/red/blue light to show if the user eats at the right speed/too fast/too slow or with short/long vibrations to inform the user that he/she eats too slow/too fast. The device can be, e.g., an eating utensil or a food carrier.

In some embodiments, the system is configured to ask a health- or goal-related question and/or to record an answer to a health- or goal-related question, at a pre-defined time or randomly, or any combination thereof. For example, the device can be an eating utensil or a food carrier 25 that is configured to count the number of bites, and can be configured to ask and/or record the user's satiety level after each bite. The question can be asked via known methods, e.g., via audible or visual signals such as, e.g., a LCD or a speaker, or via communication protocols to infer data from a component or 3rd party device, such as wired or wireless data communication. The answer to the question can be provided (i) automatically using an integrated component or a 3rd party device that automatically measures the satiety level of a user, such as, e.g., an implantable device measuring levels of hormones, peptide YY (PYY), ghrelin, or Cholecystokinin (CCK), or (ii) manually using an integrated component or a 3rd party device that is configured to receive input from a user, such as, e.g., a touch LCD screen that asks the user to rate his/her level of satiety by selecting an answer from a list of choices or by pointing out on a graphic scale, diagram, picture, or graph the right answer.

In some embodiments, the health management system 10 includes a device used as a food carrier 25 or as an eating utensil configured to regulate eating behaviors by triggering a component that is configured to send tactile, audible, or visible signals such as, e.g., a LED, a speaker or a vibrator, or configured to detect food related events by triggering a component that is configured to capture tactile, audible, or visible signals such as, e.g., a button, a microphone, a touch screen, a microphone or a camera. For example, a user can regulate his eating speed by taking a bite each time a vibration is sent by the device/fork, which can be done at preset intervals. As another example, a user can press on a button located on the device/fork to signal each time he/she taking a bite or to signal the beginning or end of the chewing time or to signal the beginning or end of a meal or to signal his satiety level. Different signal patterns can be pre-recorded to easily identify the corresponding events (e.g., pressing one time signals the start of taking a bite and pressing two times signals the end of chewing the food). Concurrently, the device records (in an internal memory 32 or in an external memory via a communication link) the date and time stamps of the events for later processing or analysis, e.g., on a smartphone application or a website.

### Labeling and Bookmarkinq

In some embodiments, the health management system 10 includes a labeling functionality and a bookmark functionality to identify, define, refine, or bookmark an activity/behavior and data associated with the activity/behavior. A device monitoring (and/or regulating) any physical activity (such as, e.g., jogging, walking, weight lifting) or any non-physical activity (such as, e.g., napping, watching TV) or any behaviors (such as, e.g., eating behaviors like eating speed) or any characteristics of an activity/behavior (such as, e.g., the ingredients information on the food currently being eaten, lifting 5 pounds, doing pushups) or any combination thereof is configured to receive an input from a user at any time during the use of the device or to prompt to receive an input at the detection of a change of behavior/activity, or any combination thereof.

This functionality is an activity/behavior labeling and/or bookmark system that can be triggered by the device itself (e.g., the device algorithm detects a change in the data pattern and prompt for input) or by a component of the device (e.g., a user presses a button or taps two times on device which is recognized by the accelerometer) or by 3rd party such as an associated website or an associated software (e.g., once the device synchronizes with the website/software, the website/software prompts to label one or more of the activity/behavior time intervals). The device input can be received through an audible signal (such as, e.g., a microphone), a visual signal (such as, e.g., a LCD screen or any display screen) or a tactile signal (such as, e.g., detecting two user's taps via an accelerometer), or any combination thereof. The input can either be selected from a pre-recorded list of text, picture, sound or vibration patterns (e.g., choosing the input from a set of pictures such as picture describing one “eating pasta” or “vacuuming” or “biking”, or saying a pre-recorded word such as “eating pasta” or “vacuuming or “biking”, or choosing the input from the list names, or tapping two times on the device to select “vacuuming” or tapping three times to select “biking”).

The device can also receive customized inputs such as a picture or video taken in relation to the input, e.g., taking a picture of the pasta plate while eating pasta taken from the device integrated camera, or such as a voice message recorded in relation to the input, e.g., recording via the device integrated microphone a memo explaining the number of sets of weight lifting the user is doing (e.g., “I lifted 20 times 15 pounds then 10 times 20 pounds”) or explaining the activity the user is doing (e.g., “I have done 2 series of 5 pushups” or “I am eating pasta with olive oil and bacon”). Indeed this feature can be very useful when a user is assessing his/her day and wants to recall events that he/she has done. Moreover this feature allows the system to assess or estimate accurately the activity/behavior by adding extra information that can be used later by the user, an algorithm or a 3rd party, e.g., to refine the estimated calories in real-time or post-processing (i.e., e.g., knowing that you are biking at a specific time allows the device to accurately estimate/interpret the calories from the accelerometer data) or to personalize the daily activity records or to provide a tailored weight management plan. Each input is recorded with its associated date and timestamp.

For example, the user can at any time press a button on the device to add a bookmark (i.e., a flag with timestamp) for latter processing. Then once at home the user can conveniently connect his/her device to a computer for synchronization and access his/her data on the website or the software application. The user can then see the list of bookmarks the user entered during the day and easily assign additional information to the bookmark event, such as defining or refining the monitored activity/behavior (e.g., by assigning a bookmark such as “eating appetizer” and adding ingredients information, another as “eating dessert”, another as “vacuuming”, another as “mowing the lawn”, another as “dancing”, etc.). Similarly the user can at any time say a word or phrase (e.g., saying, “bookmark”) or tap a pattern of vibrations on the device (e.g., tapping 5 times rapidly on the device where 5 rapid taps is triggering the device to add a bookmark event) to signal to add/record a bookmark event. In another example, the user can press a button on the device without being prompted and automatically choose from a pre-recorded list on the device to accurately choose his/her current activity.

In some embodiments, the device can prompt to enter additional information when it detects a change in the monitored data, or the device can add automatically a bookmark at the detection of such change with or without prompting the user. The device may detect that the recorded data go from a slow activity to a more intense activity and may prompt the user to enter his/her former and new current activity (e.g., asking to select walking as former activity and jogging as current activity).

In some embodiments, the health management system 10 provides an associated website that the user can log into to select from a comprehensive list of activities/behaviors the ones the user performs often. During synchronization with the website/software, the device records the user-selected activities/behaviors to propose offline any of the selected activities/behaviors as an input selection. This allows the system to load on the device only the user-selected activities/behaviors and not overload the device. If the user wants to select an activity/behavior directly on the device and the user's input is not pre-recorded on the device (i.e., the choice list proposed on the device does not include the current user's activity) then the user can create a new entry in the list (e.g., adding a new text label, a new logo or picture, tagging the new entry with a voice message or a picture).

### Real-Time Analysis

In some embodiments, the health management system 10 includes a device capable of monitoring activity behaviors or receiving monitored data (such as, e.g., calories burned, number of steps, distance walked, and so forth) from an activity monitor configured to estimate in real-time the associated eating behaviors (such as, e.g., calories consumed, eating speed, number of bites per minute, number of grams eaten per minute, amount of food that the user needs to consume based on the activity pattern, and so on) that the user needs to follow in order to maintain an optimized overall energy balance.

As an example, consider, how caloric restriction is typically measured. The Harris-Benedict and the Mifflin-St Jeor equations provide an estimate of the Basal Energy Expenditure (BEE), also called the Resting Metabolic Rate (RMR), or Basal Metabolic Rate (BMR). Predictive energy equations are routinely used in hospitals and nutrition clinics to determine the calorie requirements of various patients. Of the four most commonly used predictive energy equations, the Mifflin-St Jeor equations give the most reliable results. The Mifflin-St Jeor equations are:

| | |
|---|---|
| Male: | BMR = 10 weight + 6.25 height − 5 age + 5 |
| Female: | BMR = 10 weight + 6.25 height − 5 age − 161 |

These equations require the weight in kilograms, the height in centimeters, and the age in years. To determine total daily calorie needs, the BMR has to be multiplied by the appropriate activity factor, as follows:

1.200 = sedentary (little or no exercise)
1.375 = lightly active (light exercise/sports 1-3 days/week, approx.
590 Cal/day)
1.550 = moderately active (moderate exercise/sports 3-5 days/week,
approx. 870 Cal/day)
1.725 = very active (hard exercise/sports 6-7 days a week, approx.
1150 Cal/day)
1.900 = extra active (very hard exercise/sports and physical job,
approx. 1580 Cal/day)

Therefore, predictive energy equations depend on the selection of a level of activity by either the user himself or a third party such as a physician or nutritionist. It is therefore recognized by experts that activity levels be accounted for when calculating the ideal energy intake of an individual. Nevertheless, calculations are based on an activity factor that is static, i.e., the chosen activity level depends on the activity estimate of the user or a third party (e.g., some calculators ask the level activity of the user during the past 30 days) and therefore does not account for any daily, weekly, or monthly fluctuations and lifestyle changes. For example, if an athlete, who selected “extra active” while calculating the calories he can consumed during the day, is now taking an activity break due to vacation or a small injury then the athlete cannot continue to eat as if he/she was still “extra active,” unless he/she wants to gain weight.

The health management system 10 avoids this problem by analyzing activity behaviors of an individual in real-time to dynamically adapt his/her eating behaviors and recommendations. For example, a device that records a user's activity and estimates in real-time the calories burned is configured to calculate the new calorie requirements of the individual (i.e., his/her calories target he/she can consumed during the current day) each time the activity data is received. Based on the activity monitor readings, the activity factor used in the predictive energy equations is automatically generated and therefore the calories an individual can consume are automatically generated. Moreover, since the calorie balance is based on the calories burned versus the calories consumed, the health management system 10 can dynamically calculate an ideal (or recommended) calorie balance that one needs to maintain (in addition to the ideal calories to be consumed).

The health management system 10 is not limited to calorie calculation. The same can also be applied to behaviors. For example, a device capable of counting a number of steps or a distance travelled by a user can be configured to dynamically provide updated targets/goals of eating behaviors such as, e.g., eating 25 grams per minute or eating a bite each minute. A user with bulimia eating disorder can therefore be recommended to eat more slowly than previously recommended to limit his/her caloric intake as soon as he/she is monitored as doing very limited activity. Similarly, a user with anorexia eating disorder who was sedentary or lightly active and now is monitored as doing moderate or intense activity can therefore be recommended to eat faster than initially recommended to increase his/her caloric intake.

### Alerts/Reminders

In some embodiments, the health management system 10 includes a reminder or alert unit to remind or alert of one or more (pre-determined or not) specific events (e.g., brushing teeth, washing hands, taking antibiotics, blood glucose test for diabetics, etc.) when a time-related event occurs, when an activity generated event occurs, when a position generated event occurs, or at a specific time before, during, or after a food intake event (such as, e.g., meal time, snack time, the start or end of a meal, a specific time elapsed since the previous meal, a specific time remaining until a next scheduled meal, or when taking a bite of food), or before, during or after an activity event (such as, e.g., exercising, jogging, the beginning or end of an activity), or before, during or after an non-activity event (such as, e.g., sleeping, napping going to bed or waking up), or any combination thereof. For example, the device may remind or alert the user that it is time for a snack, to eat dinner, to take pills or medicine one hour before a meal, to take a daily glucose self-test before meals or upon waking up or at bed time or before or after exercise, drinking a liquid such as a full glass of water 30 minutes before a meal, taking supplements at beginning of lunch, doing grace or prayer of gratitude before starting meal, listening to suggestion messages before or during a meal, listening to a health-related advice and taking action such as washing hands before meals for kids or brushing teeth after a meal, making a phone call or emailing or sending a SMS to a relative or friend at the beginning of meal, and so forth. The reminder or alert may be sent directly from the device or indirectly from another third party device to another device of the user (e.g., a mobile phone).

The device may be (or may be integrated into) any type of device, e.g., an eating utensil, a food carrier, a monitoring device such as a physical activity monitoring device or a non-activity monitoring device (such as a sleep monitoring device), or a blood glucose meter device. For example, the device can be an eating utensil that counts the number of bites and records food intake duration and time and that is capable of detecting the beginning and end of a meal, an activity monitor that measures the energy spent and therefore knows the beginning and end of an exercise, a non-activity monitor that measures the quality and time a user sleeps and therefore knows the beginning and end of sleep, or a blood glucose meter that knows the time of the last blood glucose test, or any combination thereof. As another example, the device may be a computer, a server, or a CPU-enabled device that contains the information of such events and reminds or alerts users when needed. The reminders or alerts can be transmitted via signals such as, e.g., visible, vibration, or audible signals, or via known or unknown message carrier, such as, e.g., email, SMS, web, chat, tweeter message, instant message, or phone call, or any combination thereof.

In some embodiments, the system includes a device capable of directly monitoring data or indirectly receiving monitored data from a third party is configured to dynamically and adaptively provide reminders based on such data. Data can be, e.g., health-related data, activity-related data, sleep-related data or eating-related data. Reminders are traditionally static in nature. Typically, a user has to enter his/her reminders manually and plan for the schedule and event time. For example, healthy recommendations could be eating between 2.5 to 5 hours after the previous meal, drinking water more than 30 minutes before or after a meal, or taking diabetes pill one hour before a meal, and so forth. Therefore, setting a reminder to eat breakfast between 8 am to 9 am and a lunch between 11 am and noon and a reminder to drink water between 10 am and 10:30 am and another reminder to take a pill at 10:30 am is not an effective approach as any change in the schedule will make obsolete all the following reminders (e.g., if the user eats his/her breakfast at 10 am then the next meal reminder needs to be updated and consequently his pill and drink reminders). Therefore, there exists a need to have reminders that are dynamic and adaptive to the dependent events. Hence, effective reminders cannot be static and cannot be planned, as they are dependent of other events that may change during the day.

The health management system 10 introduces a notion of dynamic and adaptive reminders that take into account the dependency between reminders. Therefore, a device can appropriately reschedule recorded reminders based on events received from a device sensor or a 3rd party.

As an example, a user can setup a reminder with the following customizable fields:

```
a.  <Reminder Name>
b.  “Start Time”:
    i.  <number of minutes or hours> “minutes” (or “hours”)
“before” (or “after”) “start” (or “end”) of <DEVICE INPUT EVENT>
(i.e., e.g., “meal” or “specific activity” or “sleep”)
c.  “End Time/Date”:
    i.  <number of minutes or hours> “minutes” (or “hours”)
“before” (or “after”) “start” (or “end”) of <DEVICE INPUT EVENT>
(i.e., e.g., “meal” or “specific activity” or “sleep”)
```

Where <DEVICE INPUT EVENT> corresponds to an event directly or indirectly collected or monitored by the device, i.e., the beginning or end of an event detected or received by the device such as, e.g., an eating-related event, a sleep-related event, or an activity-related event. If the device is a utensil capable of monitoring bites and meal duration together with sleep and physical activity, then the <DEVICE INPUT EVENT> can be, e.g., the beginning/end of the meal or when the user is going to bed/waking up or when the user is starting/finishing intense physical activity or any other data collected by the device (e.g., via sensors or buttons or touch screen).

Therefore using the same example provided above, the reminders would be set, e.g., as follows: Reminder #1: Lunch; Start Time: 4 hours after end of “BREAKFAST”; Reminder #2: PM Snack; Start Time: 2.5 hours after end of “LUNCH”; Reminder #3: Drink Glass of Water; Start Time: 30 minutes after end of “ANY MEAUSNACK”; End Time: 30 minutes before start of “ANY NEXT MEAL IN SCHEDULE”; Reminder #4: Take Diabetes Pill; Start Time: 1.5 hours before start of “NEXT MEAL IN SCHEDULE”; End Time: 1 hour before start of “NEXT MEAL IN SCHEDULE”

In the above example, “any meal/snack” represents any breakfast, lunch, diner, AM snack, PM snack, or evening snack and “next meal in schedule” is referring automatically to the next meal in the schedule. In addition, reminders can have the typical customizable field found in static reminders such as date and time range of the meeting, the duration of the meeting, date range for which the reminder is kept active (start and end date of the reminder), the recurrence pattern (i.e., daily, weekly, monthly, or yearly; e.g., recur every 2 weeks on Mondays and Thursdays). Reminders can include any signals such as, e.g., visible, vibration or audible signals, and any media type such as, e.g., text, video, or sound, and any communication protocol, such as, e.g., email, SMS, or phone.

Moreover, the start or end time in the reminder can be automatically setup or automatically adjusted by the device based on the device information/data (e.g., the size of the meal that the user is eating). For example Reminder #1 stipulates to eat lunch 4 hours after end of breakfast, but the time can be automatically adjusted by the device to 3 hours (instead of 4 hours) if the user ate a small amount of food during his breakfast or to 5 hours (instead of 4 hours) if the user ate a lot of food during his breakfast depending on the data collected on the device. This automatic refinement of the time entered in the reminder is possible by, e.g., assessing the number of bites from the previous meal by a eating monitoring utensil, or assessing the weight of each bite and therefore of the meal by a eating monitoring utensil, or assessing the length of meal by inferring from first to last bite, or assessing the picture of the previous meal taken by a camera on utensil, or assessing the food ingredients eaten by the user where the ingredients are collected on a database by a user input component or a 3rd party. Moreover, the time entered in the reminder can be automatically setup using the information collected from the device sensors or database (e.g., a physician or nutritionist can access user's data, assess the correct settings or time of reminders, and send back the appropriate time of the reminder to change/set it on the server).

Implantable Extensions

In some embodiments, parts or the whole health management system 10 is implanted in a human body, such as, for example, a wrist or a tooth. Implanted devices may include any sensor 35 described in this invention or in the U.S. patent application Ser. No. 12/408,622, such as, for example, microphone, accelerometer 38, optical sensors 36 or proximity sensors. For example, some sensors can be integrated in a dental crown 40. A crown with a microphone can detect chewing pace by monitoring sounds of jaws that chew on food, or a crown with an accelerometer can monitor jaw motion and infer bites. The dental crown device 40 can be either a stand-alone device or an extension 21 that communicates via data links 26 with the enclosure 20.

The implanted extension 21 can include any of the electrical circuits described in U.S. Provisional Patent Application No. 61/359,378 entitled “Dynamic Scale and Accurate Food Measuring,” and filed on Jun. 29, 2010, which is hereby incorporated by reference.

It should be understood that the present disclosure is not limited to the embodiments disclosed herein as such embodiments may vary somewhat. It is also to be understood that the terminology employed herein is used for the purpose of describing particular embodiments only and is not intended to be limiting in scope and that limitations are only provided by the appended claims and equivalents thereof.

I/We claim:

1. An electronic device for managing a user's health, the device comprising:
an enclosure comprising one or more electrical circuits including:
one or more sensors configured to detect health related activity of the user;
a data link unit configured to communicate with other devices, including at least one extension having a defined interface to receive additional sensory information related to user health;
a power source configured to provide power to the one or more electrical circuits;
a control panel configured to receive input from the user;
a user interface configured to display output to the user;
a processor unit configured to execute instructions for analyzing sensory input received from the one or more sensors to take health-related actions; and
a memory configured to store data and instructions accessible by the processor unit.

2. The device of claim 1 wherein one of the sensors and the instructions executed by the processor unit are further configured to detect the characteristics of the user.

3. The device of claim 1 wherein one of the sensors is further configured to detect a load and the characteristics of the device's load before, during or after being loaded.

4. The device of claim 1 wherein at least one of the sensors is an optical device.

5. The device of claim 1 wherein one or all of the electrical circuits are implanted in a human body part.

6. The device of claim 1 wherein one of the sensors is further configured to detect the tare or the weight of a load even while the device is in motion.

7. The device of claim 1 wherein one of the sensors is further configured to locate the device geographical position.

8. The device of claim 1 wherein one of the sensors is further configured to detect the user's sleep cycles.

9. The device of claim 1 wherein one of the sensors is further configured to detect humidity levels.

10. The device of claim 1 wherein one of the sensors is further configured to detect the motion or non-motion of the device.

11. The device of claim 1 wherein at least one of the control panel, one or more sensors, and the instructions executed by the processor unit are further configured to bookmark and label the activity or behaviors of the user.

12. The device of claim 1 wherein the instructions executed by the processor unit are configured to update in real-time the recommended or ideal energy balance and behaviors.

13. The device of claim 1 wherein the instructions executed by the processor unit are configured to alert the user using dynamic and adaptive reminders.

14. The device of claim 1 wherein the instructions executed by the processor unit are configured to setup or adjust reminders.
